(19) United States
(12) Patent Application Publication
(10) Pub. No.: US 2024/0253056 A1
(43) Pub. Date: Aug. 1, 2024

(54) CLEANING DEVICE FOR CLEANING A GAS

(71) Applicant: Markus Villinger, Telfes (AT)

(72) Inventor: Markus Villinger, Telfes (AT)

(21) Appl. No.: 18/430,169

(22) Filed: Feb. 1, 2024

Related U.S. Application Data
(63) Continuation of application No. PCT/AT22/60269, filed on Aug. 2, 2022.

(30) Foreign Application Priority Data
Aug. 2, 2021 (AT) A 50630/2021

(51) Int. Cl. B03C 3/68; A61L 9/22; B03C 3/011; B03C 3/02; B03C 3/155; B03C 3/36; B03C 3/41; B03C 3/45; B03C 3/53; B03C 3/64; B60H 3/00; F24F 8/192

(52) U.S. Cl. CPC B03C 3/68; A61L 9/22; B03C 3/011; B03C 3/025; B03C 3/155; B03C 3/368; B03C 3/41; B03C 3/455; B03C 3/53; B03C 3/64; B60H 3/0078; F24F 8/194; A61L 2209/111; A61L 2209/14; B03C 2201/10; B03C 2201/24

(57) ABSTRACT

The invention relates to a cleaning device for cleaning a gas, in particular air, comprising: a first electrode which is populated with at least one element, preferably a plurality of elements, said at least one element having a surface with a plurality of tips and/or protrusions in order to generate a non-homogeneous electromagnetic field, a second electrode which preferably lies opposite the first electrode, and a voltage source which is electrically connected to the first electrode and/or the second electrode and by means of which a voltage can be built up between the first electrode and the second electrode.

The present invention relates to a cleaning device for cleaning a gas, in particular air, as well as the arrangement of such a cleaning device in a ceiling panel and/or a wall covering and the use of such a cleaning device.

Corresponding cleaning devices are used to remove contaminants from gases—preferably air—wherein particular importance is attached to the fact that particles, viruses, bacteria, microbial constituents and also contaminated aerosols or droplets are removed from the gas or likewise corresponding bacteria or viruses in the gas are killed (and thus a contamination of the gas is neutralized).

Such cleaning devices are for example known through the state of the art through filtration systems, wherein a gas stream is guided through a filter, wherein particles up to a particular minimum size can be removed by the filter. However, corresponding filter systems reach their limits above all in the case of particles with a size of <0.3 μm, such as e.g. viruses and bacteria, which are carried in the gas freely or in aerosols, in particular liquid droplets.

Thus, it is for example known through AT 196979 B to use ionizing filters, in which the particles to be separated from a gas are charged in a unipolar manner as a result of an ionization of the gas brought about by self-sustaining gas discharge, wherein an electrode system serves to charge the particles.

Thus, it is known to charge the particles to be separated from the gas—thus liquid droplets or dust particles—by guiding the gas through an electric field, in which corona discharges take place as a result of field enhancements at tips or elevations of the electrodes, wherein ions are produced, which accumulate in part on the particles to be separated, as a result of which the particles to be separated can be removed by an electrostatic separator.

However, a disadvantage here is that due to the “peak” voltages generally used corona discharges and thus ozone and nitrogen oxides are produced, which are then present in the cleaned gas and are harmful to health.

A further development of such an ionizing filter is described by AT 377204 B. Here, it is discussed that via the use of several series resistors the corona discharges at the tips or projections of the electrodes can be reduced or prevented, as a result of which accordingly the production of nitrogen oxides and ozone can be reduced.

The object of the present invention is to provide a cleaning device for cleaning a gas, with which contaminants, particles, bacteria or viruses can be removed from the gas more effectively than in the state of the art, without adding toxic substances, such as for example ozone or nitrogen oxides, to the gas.

This object is achieved by a cleaning device for cleaning a gas, in particular air, with the features of claim 1 as well as by the arrangement of a cleaning device according to the features of claim 21 and by the use of a cleaning device corresponding to the features of claim 22.

According to the invention it is provided that a cleaning device for cleaning a gas, in particular air, comprises the following:
a first electrode, which is populated with at least one body, preferably a plurality of bodies, wherein the at least one body has a surface with a plurality of tips and/or projections for generating an inhomogeneous electromagnetic field,
a second electrode, preferably lying opposite the first electrode, and
a voltage source electrically connected to the first electrode and/or the second electrode, with the aid of which voltage source a voltage can be built up between the first electrode and the second electrode.

Through the arrangement of the first electrode relative to the second electrode and the provision of the voltage source which generates a voltage between the electrodes, an inhomogeneous electrostatic field can be generated between the electrodes, as a result of which the gas present between the electrodes can be cleaned.

Thus, an electric dipole is generated in the undesired constituents, for example in the form of particles, due to the imposed voltage at the tips and/or projections of the body arranged on the first electrode, as a result of which the particles present in the gas are either attracted or repelled (depending on their existing charge). Through the inhomogeneous electric field, the undesired constituents are preferably moved in the direction of the first electrode (dipole in the inhomogeneous electric field).

Through the attraction of the particles to the first electrode, the particles can attach to the first electrode and/or can be supplied with a voltage.

Through the repulsion of the particles by the first electrode, the particles can be conveyed to the second electrode, wherein they can attach to the second electrode or can be supplied with a voltage.

If the particles now attach to the first and/or the second electrode, they can be effectively removed from the gas.

Through the supply of a voltage, bacteria or viruses present on the particles or in the particles can moreover be killed.

Cleaning devices according to the invention can be used to remove contaminants from gases—preferably air—in particular to eliminate particles, viruses, bacteria, microbial constituents and also contaminated aerosols or droplets, in particular smaller than 0.1 μm, from the gas or likewise to kill corresponding bacteria or viruses in the gas (and thus to neutralize a contamination of the gas).

Through a corresponding design of the invention, a much higher electrical voltage can be applied to the electrode and thus higher efficiency than in conventional methods can be achieved, without building up corona discharges, as a result of which the pollution of the gas with ozone or nitrogen oxides is also prevented.

The voltage applied between the first electrode and the second electrode can preferably be chosen such that no corona discharge occurs. Tests by the applicant have shown that this voltage can depend on the temperature and/or the humidity and/or the pressure and/or loading of the gas, in particular with aerosols, of the gas to be cleaned. Suitable voltages can be determined using tests.

Tests which the applicant had carried out by Aerosol Research & Engineering Labs (Kansas, USA) have shown that the invention cleaned contaminants in the examined gas up to below the detection limit (of 99.99992%)—within half the usual time for tests on such devices.

Advantageous embodiments and example variants are defined with reference to the dependent claims.

It can be provided that the first and the second electrode are formed cylindrical and arranged concentrically.

The voltage can be a direct or alternating voltage. This voltage can be supplied in a uniform or pulsating manner. A temporary or permanent reversal of the polarity can be used to counteract a fouling of the electrode due to accumulation of particles.

It can preferably be provided that a control or regulating device is provided, which is formed to vary the voltage applied between first electrode and second electrode, with the result that in the case of a change in temperature and/or the humidity and/or the pressure and/or loading of the gas, in particular with aerosols, preferably no corona discharge forms at tips and/or projections.

It can be provided that the control or regulating device is formed to vary a cleaning capacity of the cleaning device taking into account a temperature and/or humidity and/or ozone content and/or level of loading and/or pressure of the gas—preferably using a temperature and/or ozone and/or particulate and/or pressure and/or humidity sensor connected or connectable to the control or regulating unit.

It can preferably be provided that the regulating device is formed to control or regulate the cleaning capacity of the cleaning device by varying at least one of the following: a voltage applied between first electrode and second electrode, change in the polarity of the voltage, deactivation of one or more electrode pairs, if several electrode pairs are present, variation of a distance between the first and the second electrode, cooling or heating of the gas to be cleaned, change in a flow rate of the gas to be cleaned between the first electrode and the second electrode, change in a humidity of the gas to be cleaned.

Due to temperature and/or humidity and/or pressure and/or loading of the gas, the stress state in which a corona discharge would form at the tips and/or projections changes, as a result of which the control or regulating unit, taking these into account a corresponding variation of the voltage can be carried out, in order for example to prevent corona discharges, and/or a desired cleaning capacity can be set.

It can preferably be provided that the voltage source is formed to supply the first electrode and/or the second electrode with 30 μA, in particular that no electric currents that are life-threatening for humans occur.

It can be provided that the voltage source is formed to supply the first electrode and/or the second electrode with a voltage of 0.5 kV.

It is preferably provided that the voltage source is formed to supply the first electrode and/or the second electrode with an—in particular pulsating—direct voltage or an alternating voltage—preferably with a frequency greater than/equal to 1 Hz.

It can preferably be provided that a temporary reversal of the polarity is carried out, in particular in order to counteract a fouling of the electrode due to accumulation of particles.

It can preferably be provided that the first electrode is supplied with a voltage and the second electrode is preferably grounded and/or the second electrode is supplied with a voltage and thus the required potential difference is provided.

It can be provided that the at least one body populating the first electrode is formed by a non-conducting or weakly conducting material—preferably polyamide.

It can preferably be provided that the at least one body populating the first electrode has a plurality of—preferably vertically—protruding electrode fibers or electrode rods.

It can be provided that the electrode fibers and/or the electrode rods have a length of from 0.5 to 40 mm, preferably 1 to 20 mm, particularly preferably 1 to 5 mm.

The electrode fibers and/or electrode rods can preferably be distributed over the surface area of the first and/or second electrode and/or applied in vertical orientation, particularly preferably by flocking.

It can be provided that the at least one body—preferably the electrode fibers and/or the electrode rods—has a coating using a non-conducting coating material, particularly preferably Teflon®.

Through a coating of the at least one body and/or the electrode fibers and/or the electrode rods it can be provided that the at least one body has particularly good properties with respect to cleaning, wherein deposits do not adhere to the surface and can for example be washed off using a cleaning liquid, preferably water.

It can be provided that the coating arranged on the at least one body—preferably on the electrode fibers or electrode rods—is formed to expose the base material at tips and/or projections opposite the second electrode. In other words, tips of the electrode fibers or electrode rods thus remain free of the coating, as a result of which the inhomogeneity of the electric field is increased.

It can be provided that the electrode fibers and/or electrode rods arranged on the at least one body are formed from a polyamide material.

It can preferably be provided that the electrodes are formed from an electrical, conductive base material and/or consist of a non-conductive base material, which preferably has a conductive coating.

It can be provided that a further coating, which is preferably formed by a non-conducting coating material that differs from the coating, particularly preferably a polymer plastic, is provided on the tips and/or projections of the at least one body—in particular the electrode fibers and/or electrode rods.

It can be provided that the coating material of the further coating (of the tips and/or projections) differs from the coating material of the coating (the base body of the at least one body) through an electrical conductivity and/or a capacity to absorb liquids.

Thus, it can be provided, for example, that the further coating can absorb 200% more moisture, preferably water, than the coating.

The capacity of the further coating to absorb moisture, in particular water, can lie in a range of from 1% to 5%, preferably 2.5% to 3%.

The capacity of the coating to absorb moisture, in particular water, can lie in a range of from 0.05% to 0.3%, preferably 0.1%.

It can preferably be provided that the second electrode extends along the first electrode at a defined distance, wherein a flow channel for the gas is formed by the defined distance between first electrode and second electrode.

It can preferably be provided that the defined distance between the first and the second electrode can be set by the control or regulating unit.

For this purpose, an actuator can for example be provided, which is connected or connectable in a signal-carrying manner to the control or regulating unit, as a result of which the distance between first and second electrode can be varied.

It can preferably be provided that the defined distance between the first and the second electrode is between 0.5 and 50 cm, preferably between 1 and 20 cm, particularly preferably between 2 and 5 cm.

An electrically non-conducting layer which collects moisture, droplets and/or particulate matter (for example a cellulose ply, a paper ply) can be arranged between the first and the second electrode. This layer can preferably be formed replaceable.

An embodiment can be provided, wherein the second electrode has been or is realized by a grounded environment.

It can preferably be provided that at least one guide device—preferably having a fan—is provided, in order to guide the gas to be cleaned through the flow channel between the first and the second electrode.

It is preferably provided that to actively promote the flow of the gas between the first and the second electrode a fan is provided, which can for example be operated with a noise level of from 0.1 to 200 dB, wherein a noise level below 50 dB would particularly preferably be implemented.

It can be provided that the plurality of tips and/or projections stand out from the first electrode in the direction of the second electrode with a length of between 0.5 and 40 mm, preferably 1 and 20 mm, particularly preferably 1 and 5 mm.

This length can preferably correspond to an electrode fiber length of the electrode fibers and/or the electrode rods.

It can be provided that the plurality of tips and/or projections extend from the first electrode in the direction of the second electrode with different lengths.

Through the design of the tips and/or projections with different extension lengths, the inhomogeneity of the electric field can be supported, as a result of which the cleaning efficiency of the cleaning device can additionally be increased.

It can be provided that the second electrode is formed by at least one liquid bath, preferably a water bath.

It can be provided that the second electrode is formed by at least one liquid bath, preferably a water bath, and is grounded or supplied with a voltage.

Through the design of the second electrode using a liquid bath, particles from the gas to be cleaned striking the second electrode can be easily bound to the electrode or to the liquid.

In order to enhance the effect, it can for example be provided that the electrical conductivity of the liquid bath is influenced by additives, and/or that the surface tension of the liquid bath is reduced by additives, such as can be effected, for example, in the case of a water bath through the addition of salts, acids, soaps, oils or alcohol. Through the reduced surface tension it can be provided that particles separated from the gas sink in the liquid bath and preferably accumulate on a base of the liquid bath (wherein the separated particles would possibly float on the surface in the case of increased surface tension of the liquid bath) and/or can be better bound by the liquid.

Furthermore, the design of the second electrode as a liquid bath results in the advantage of a simple implementation of the cleaning of the second electrode, by replacing the liquid. Such a cleaning by liquid replacement can for example be effected in an automated manner.

Furthermore, the design of the second electrode as a liquid bath results in the advantage of a simple implementation of the cleaning of the second electrode, wherein the liquid preferably circulates (for example through a pump and/or a filter). Such a cleaning by circulation through a filter system can for example be effected in an automated manner.

It can be provided that the cleaning device has a filtration device for the gas to be cleaned, preferably upstream of the electrodes in terms of flow.

A corresponding filtration device can for example have an HEPA filter and/or a ULPA filter, wherein larger particles can be removed from the gas to be cleaned by this filter device and only then is the gas to be cleaned led to the cleaning device (or more precisely: between the electrodes).

It can be provided that the cleaning device has a heating device, preferably wherein the control or regulating unit is formed to control or regulate a heating capacity of the heating device.

Through a heating device, an increase in efficiency can be achieved by heating the first or second electrode of the cleaning device. It can particularly preferably be provided that the first and/or the second electrode is heated to a temperature of at least 40° C., preferably 90° C. to 100° C., by the heating device. Here, a direct heating of the electrodes can be implemented or the electrodes are heated indirectly via a heating of the gas to be cleaned.

It can preferably be provided that the heating device has at least one heating layer, which at least one heating layer is arranged on the tips and/or projections of the at least one body—in particular the electrode fibers and/or electrode rods—and is formed to control the temperature of the tips and/or projections of the at least one body with the aid of the voltage applied to the first electrode.

It can be provided that the at least one heating layer is arranged between the further coating of the tips and/or projections and the base material of the at least one body, preferably directly on the base material.

The at least one heating layer can for example be a layer—preferably applied flat—with a layer thickness of from 0.001 mm to 2 mm, in particular 0.008 to 0.05 mm.

It can be provided that the control or regulating device is formed to vary a heating capacity of the heating device, in particular of the at least one heating layer, taking into account a temperature and/or humidity and/or ozone content and/or level of loading and/or pressure of the gas—preferably using a temperature and/or ozone and/or particulate and/or pressure and/or humidity sensor connected or connectable to the control or regulating unit.

An electrical conductivity of the tips and/or projections can thus be controlled and/or regulated, taking into account the environmental influences, by the control or regulating device through variation of the heating capacity of the heating device, in particular of the at least one heating layer, in order to avoid and/or to be able to prevent corona discharge and/or plasma discharge at tips and/or projections. A corona discharge and/or plasma discharge at tips and/or projections would namely in turn result in ozone and/or nitrogen oxide formation, as a result of which the gas to be cleaned would be contaminated again.

It can preferably be provided that the control or regulating unit is formed to heat, in a first operating mode, one or more electrodes in order to increase the efficiency, wherein, after a selectable period of time, switching into a second operating mode is effected, in which the electrode, or electrodes, is no longer heated, with the result that although the efficiency drops, the energy requirement is likewise reduced and for example the air conditioning of the room is not affected.

By means of such an embodiment example it can for example be provided that, in a first operating mode, the gas contained in a room is transformed once, wherein, after the entire quantity of gas in the room has run through one cleaning step, switching into the second operating mode is effected, in which, after a first run of the entire quantity of gas through the cleaning device, only a lesser cleaning effect is necessary as a lower contamination of the gas to be cleaned can be expected.

It can be provided that the cleaning device has a temperature control device downstream of the electrodes in terms of flow, preferably wherein the control or regulating unit is formed to actuate or regulate the temperature control device in such a way that a gas temperature of the gas leaving the cleaning device can be set.

Thus, an air conditioning unit can for example be provided as temperature control device, which then adjusts the cleaned gas to a predefinable target temperature and for example feeds it to an interior of an office or residential building.

Naturally, in the reverse direction, it would also be possible to heat the cleaned gas in order to bring it to a desired temperature.

It can preferably be provided that a retaining device is provided in order to support the first and/or the second electrode.

This retaining device can preferably have a spring catch. Furthermore, a quick connection system can for example be provided for the voltage supply, the first and/or the second electrode, as a result of which an easy replaceability of the first and/or the second electrode can be implemented.

It can also be provided that the present invention is combined with an embodiment example of a conventional ozonizer (ozone generator) known from the state of the art, wherein for example the conventional ozonizer first cleans a quantity of gas and surfaces, wherein the ozone formed is then degraded after a half-life of, for example, 30 minutes and the procedure is preferably continued with the cleaning device according to the invention, which operates ozone-free.

Thus, for example, a gas or an air could first be fed to an interior via the production of ozone, wherein the ozone in the interior (in which naturally people must not be located) can be used for the surface cleaning and/or mold removal and the ozone formed is then degraded after a half-life of, for example, 30 minutes before people gain access to the interior.

It can be provided that the control or regulating device is connected to a warning device, wherein a warning signal can for example be output to an operator, as soon as the cleaning device is inactive—for example due to an upcoming maintenance interval and/or a fault and/or a power failure.

This warning device can operate acoustically or by means of an optical signal. The control or regulating device with a warning device can be realized by means of a wired or wireless connection, for example via computer, mobile telephone or tablet.

It is preferably provided that an electrode surface area lies in a range between 1 cm² and 100 m² and particularly preferably has a size of approx. ¼ m².

It can be provided that the electrodes face each other in parallel and/or are at an angle of ≤359° to each other and/or have the same surface area or are designed in the ratio ≤1:10, preferably are designed in the ratio 1:1.5.

It can be provided that the second electrode (counterelectrode) is placed at a particular distance with respect to the first electrode, preferably in parallel.

It can be provided that the second electrode (counterelectrode) is grounded.

It can be provided that the second electrode (counterelectrode) can be supplied with a voltage.

It can be provided that, on a side facing away from the second electrode, the first electrode has at least one further body, preferably a plurality of further bodies, which has a surface with a plurality of tips and/or projections for generating an inhomogeneous, electromagnetic field, wherein a third electrode can be arranged on the side of the first electrode facing away from the second electrode.

Correspondingly, the cleaning device for cleaning a gas could be expanded in a modular manner.

It can be provided that, on a base support, a substrate is applied which holds the plurality of bodies on the base support, wherein the plurality of bodies (for example the electrode fibers) are spaced apart from each other.

A conductive layer (for example a conductive varnish) can then be deposited between the individual bodies, which conductive layer connects the plurality of bodies to each other in a conductive manner.

A top layer can then for example also be deposited to protect the conductive layer or the plurality of bodies (for example a Teflon coating).

The substrate, which acts as a support for the plurality of bodies, can for example be formed as a semiconductor and be present in a thickness of from 0.01 mm to 10 mm, preferably 0.1 to 1 mm.

It can preferably also be provided that the counterelectrode is formed to vibrate in an ultrasonic range (for example with a frequency of from 20 kHz to 10 GHz).

It can also be provided that the invention is combined with a light source for UV-A light and/or UV-B light and/or UV-C light, in order to kill viruses or bacteria that may be present.

It can also be provided that one or both electrodes has a microbe-killing surface.

In the same way, a microwave source or a source of a disinfectant could for example be provided, wherein the disinfectant could for example be introduced into the gas to be cleaned as an aerosol and/or could be applied to the electrodes.

Protection is furthermore sought for the arrangement of a cleaning device according to the invention in a ceiling panel and/or a wall covering and/or a floor panel and/or a couch and/or a chair, wherein the cleaning device is preferably integrated into a headrest and/or the gas to be cleaned can be fed to the cleaning device via a projection.

It can be provided that the arrangement of the cleaning device is formed freestanding and/or portable.

A corresponding arrangement for example has the striking advantage that a cleaning device according to the invention can be retrofitted into existing systems, wherein an existing ceiling panel could be replaced with one with a cleaning device, without affecting the appearance of a room.

It can be provided that the cleaning device has at least one (preferably standardized) external dimension which corresponds to internationally standardized high-efficiency particulate air filters. An external dimension of 592×592×292 mm (width×height×depth) is particularly preferred.

By adapting the cleaning device to at least one external dimension of a filter system it can be provided that the cleaning device can be used in already existing filter systems, wherein the filter insert (for example an EPA/HEPA/ULPA filter) is removed from the filter system and can be replaced with a cleaning device according to the invention or the cleaning device can be removed if necessary and can be replaced with a conventional filter.

Protection is likewise sought for the use of a cleaning device according to the invention for cleaning a gas, preferably an ambient air, of at least one building interior and/or vehicle or aircraft interior.

Further application examples of the use of a cleaning device according to the invention would be for example enclosed spaces of a vehicle, an aircraft, a flying device, a watercraft, a spacecraft cabin, a space station, lifts, medical practices, operating rooms, building interiors, interiors of medical facilities or the like.

It is preferably provided that the cleaning device is used in a ceiling panel and/or a wall covering and/or a floor panel and/or freestanding or as a portable device in the case of enclosed spaces of medical practices, operating rooms, building interiors and interiors of medical facilities.

Furthermore, an embodiment is provided in which the cleaning device can be combined with a facility for lying and/or sitting, for example a dentist's chair, wherein the gas to be sterilized is sucked in by means of a suction device on one or both sides of the headrest and guided into the cleaning device.

This suction device can be designed freely movable or integrated. One embodiment also provides integrating the suction device in a protrusion (as a projection) attached to the headrest of the chair or armchair. A further embodiment uses a flexible suction tube (as a projection), which can be bent and curved. Application examples of this would be, among other things, dentist's chairs, hairdresser's chairs, or seats in public or private means of transport.

Furthermore, an embodiment can be provided in which the second electrode of the cleaning device is formed by objects and/or structures such as for example interior walls, on which, based on the principle of electrostatics, the particles, viruses, bacteria, microbial constituents and also contaminated aerosols or droplets charged by the first electrode can collect and adhere there.

Further details and advantages of the present invention are explained in more detail below by means of the description of the figures with reference to the embodiment examples represented in the figures. There are shown in:

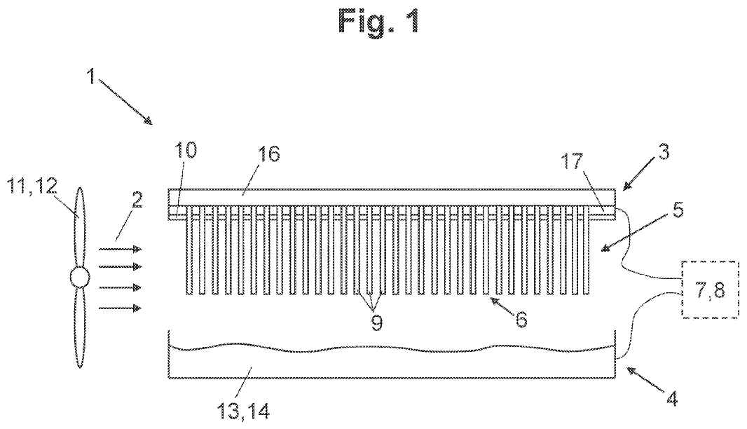

FIG. 1 a first embodiment example of a cleaning device

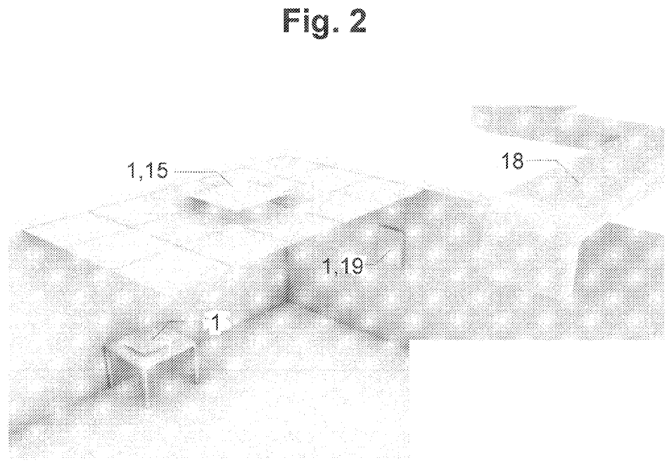

FIG. 2 various arrangement examples of a cleaning device

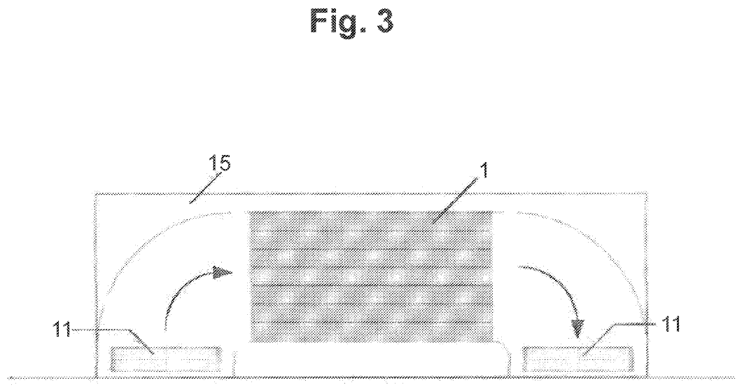

FIG. 3 a use example of a cleaning device in a ceiling panel

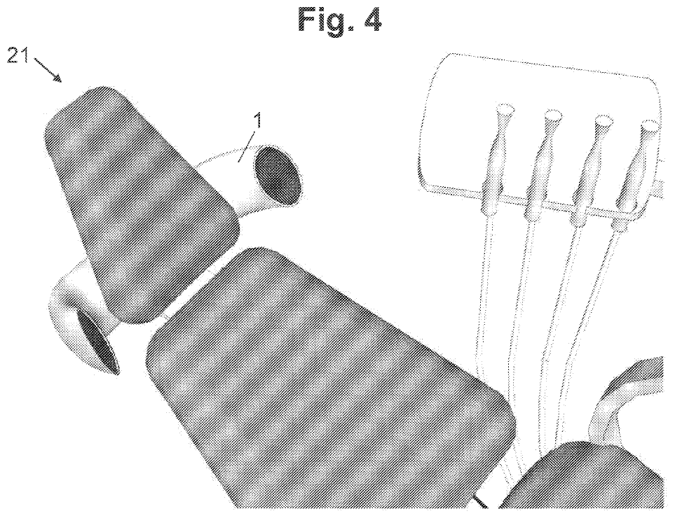

FIG. 4 an embodiment example of a cleaning device with a dentist's chair

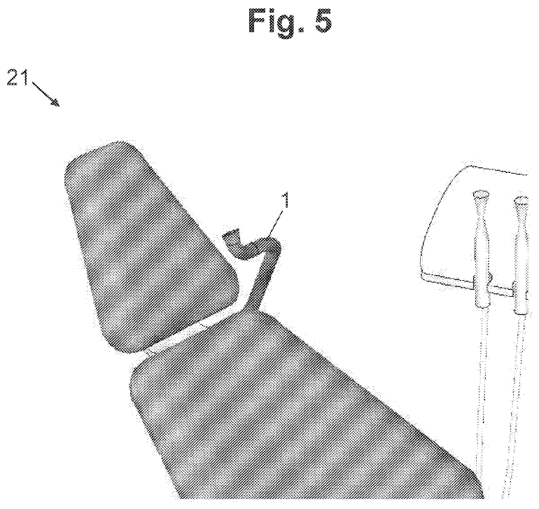

FIG. 5 a further embodiment example of a cleaning device with a dentist's chair and

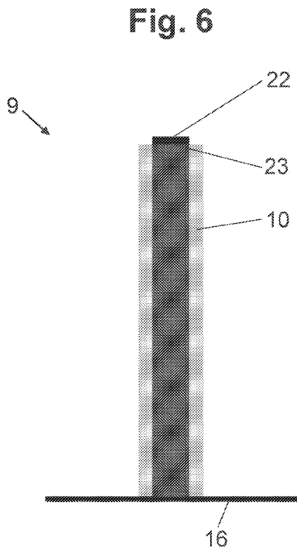

FIG. 6 an embodiment example of an electrode fiber in cross section.

FIG. 1 shows a first embodiment example according to the invention of a cleaning device 1. The cleaning device 1 has a first electrode 3 and a second electrode 4 lying opposite the first electrode 3.

In this embodiment example the first electrode 3 and the second electrode 4 are electrically connected to a voltage source 7, wherein the shown voltage source 7 of this embodiment example is coupled to a control or regulating unit 8.

The first electrode 3 of this embodiment example has a plurality of bodies 5, which bodies 5 form a surface with a plurality of tips 6 for generating an inhomogeneous, electric field.

The plurality of bodies 5 of this embodiment example of the first electrode 3 are formed as electrode fibers, which extend with a longitudinal extent from the first electrode 3 in the direction of the second electrode 4 and have a tip 6 at the end remote from the first electrode 3.

The first electrode 3 is constructed by a layer construction, wherein the electrode fibers 9 are held on a support element 16 via an electrically conductive substrate 17.

The support element 16 can be made of a non-conducting or weakly conducting material or a semiconductor, on which the substrate 17, for example in the form of a varnish which is conductive, can be deposited and the electrode fibers 9 can be embedded.

Moreover, the first electrode 3 comprises a coating 10, which has been applied to the conductive substrate 17 and surrounds the electrode fibers (not visible here).

The coating 10 of this embodiment example is implemented by a Teflon material, which covers the substrate 17 as well as the surface of the electrode fibers 9, apart from the tips 6 of the electrode fibers 9.

The tips 6 of the electrode fibers 9 are formed coating-free, in order not to influence the dipole effect of the electrode fibers 9, which is formed by the voltage build-up between the second electrode 4 and the first electrode 3 (more precisely: the substrate 17).

The electrode fibers 9 of this embodiment example are formed from a polyamide material, which is surrounded by the coating 10.

In this embodiment example the second electrode 4 is implemented as a liquid bath 13—more precisely: water bath 14—and connected to the voltage source 7.

A flow channel, through which an air 2 can flow, forms between the first electrode 3 and the second electrode 4, wherein the guide device 12 is provided with the fan 11 to promote an air flow.

During the cleaning process, a voltage is now built up between the first electrode 3 and the second electrode 4, whereby dipoles form at the tips 6 of the electrode fibers 9, which has the result that during flow through the flow channel particles, viruses or bacteria contained in the air 2 either collect on the first electrode 3 or are repelled by it into the water bath 14 (or the second electrode 4) and are received by the water bath 14.

The separated particles—if they contain bacteria or viruses—can be killed by the voltage formed at the electrodes 3, 4.

At the opposite end of the flow channel between the first electrode 3 and the second electrode 4 from the guide device 11, a cleaned air can now subsequently be removed or for example be fed into a room.

FIG. 2 shows various application examples of a cleaning device according to the invention, wherein the cleaning device 1 could for example be integrated into a ceiling panel 15 or can be used in a wall covering 19.

Furthermore, the cleaning device 1 could also be manufactured as an independent component, which can be positioned in the room (for example visible on the table represented).

The use of a cleaning device 1 according to an embodiment example according to the invention in a ventilation pipe 18 of a ventilation system of a building is also entirely conceivable.

FIG. 3 shows an embodiment example of a cleaning device 1 in connection with a ceiling panel 15.

Here, air is sucked in from an interior 20 by the ceiling panel—more precisely: by an axial fan 11—and fed to the cleaning device 1 via the guide device 12.

Through the cleaning device 1, contaminants, airborne particles, bacteria or animals can then be removed from the air 2 fed in, which can subsequently be fed to the interior 20 again via a further axial fan 11.

The use of several cleaning devices, which are arranged in series, is also entirely conceivable. A parallel arrangement of the cleaning device, wherein a lower flow rate can be chosen, is also entirely conceivable.

FIG. 4 shows an embodiment example in which the cleaning device 1 is combined with a dentist's chair 21, wherein the gas to be sterilized is sucked in by means of a suction device on both sides of the headrest and guided into the cleaning device 1.

FIG. 5 shows an embodiment example in which the cleaning device 1 is combined with a dentist's chair 21, wherein a suction device is designed freely movable.

One embodiment also provides integrating the suction device in a protrusion attached to the headrest of the chair or armchair. Alternatively, a flexible suction tube, which can be bent and curved, can suction a gas to be cleaned and feed it to the cleaning device 1.

FIG. 6 shows a cross section of an embodiment example of an electrode fiber 9 of a body 5 of a cleaning device 1 according to an embodiment example of the invention.

The electrode fiber 9 of this embodiment example is implemented by a base material in an elongate (hairlike) form. The base material is formed by a polyamide material.

The electrode fiber 9 of this embodiment example is surrounded on its lateral surface by a coating 10, wherein the coating 10 is formed to expose the tip 6 of the electrode fiber 9 opposite the second electrode 4.

In other words, tips 6 of the electrode fibers 9 remain free of the coating 10, as a result of which the inhomogeneity of the electric field is increased.

A heating layer 23, which surrounds the tip 6 in a flat way, is provided at the tips 6 of the electrode fibers 9.

The heating layer 23 can be deposited on the tip (for example using a coating method) with a layer thickness of from 0.001 mm to 2 mm, in particular 0.008 to 0.05 mm.

Through a voltage applied to the electrode fiber 9 it can be provided that the heating layer 23 is used for heating the tip 6, as a result of which a corona discharge and/or plasma discharge at the tips 9 can be avoided and/or prevented.

A corona discharge and/or plasma discharge at the tips 6 would namely in turn result in ozone and/or nitrogen oxide formation, as a result of which the gas to be cleaned would be contaminated again.

The heating layer 23 is arranged directly on the base material between the further coating 22 of the tip 6 and the base material of the electrode fiber 9.

The further coating 22 of the tip 6 is formed by a coating material that differs from the coating 10.

### LIST OF REFERENCE NUMBERS

1 cleaning device
2 air
3 first electrode
4 second electrode
5 body
6 tip
7 voltage source
8 control or regulating unit
9 electrode fibers
10 coating
11 fan
12 guide device
13 liquid bath
14 water bath
15 ceiling panel
16 support element
17 substrate
18 ventilation pipe
19 wall covering
20 interior
21 dentist's chair
22 further coating
23 heating layer

1. A cleaning device for cleaning a gas, in particular air, comprising:
a first electrode, which is populated with at least one body, preferably a plurality of bodies, wherein the at least one body has a surface with a plurality of tips and/or projections for generating an inhomogeneous electromagnetic field,
a second electrode, preferably lying opposite the first electrode, and
a voltage source electrically connected to the first electrode and/or the second electrode, with the aid of which voltage source a voltage can be built up between the first electrode and the second electrode.

2. The cleaning device according to claim 1, wherein a control or regulating device is provided, which is formed to vary the voltage applied between first electrode and second electrode, with the result that preferably no corona discharge and/or plasma discharge forms at tips and/or projections.

3. The cleaning device according to claim 2, wherein the control or regulating device is formed to vary a cleaning capacity of the cleaning device taking into account a temperature and/or humidity and/or ozone content and/or level of loading and/or pressure of the gas—preferably using a temperature and/or ozone and/or particulate and/or pressure and/or humidity sensor connected or connectable to the control or regulating unit.

4. The cleaning device according to claim 3, wherein the regulating device is formed to control or regulate the cleaning capacity of the cleaning device by varying at least one of the following: a voltage applied between first electrode and second electrode, change in the polarity of the voltage, deactivation of one or more electrode pairs, if several electrode pairs are present, variation of a distance between the first and the second electrode, cooling or heating of the gas to be cleaned, change in a flow rate of the gas to be cleaned between the first electrode and the second electrode, change in a humidity of the gas to be cleaned.

5. The cleaning device according to claim 1, wherein the at least one body populating the first electrode is formed by a non-conducting or weakly conducting material—preferably polyamide.

6. The cleaning device according to claim 1, wherein the at least one body populating the first electrode has a plurality of—preferably vertically—protruding electrode fibers and/or electrode rods.

7. The cleaning device according to claim 1, wherein the at least one body preferably the electrode fibers and/or the electrode rods—has a coating using a non-conducting coating material, particularly preferably Teflon.

8. The cleaning device according to claim 7, wherein the coating arranged on the at least one body—preferably on the electrode fibers and/or electrode rods—is formed to expose the base material at tips and/or projections opposite the second electrode.

9. The cleaning device according to claim 1, wherein a further coating, which is preferably formed by a non-conducting coating material that differs from the coating, particularly preferably a polymer plastic, is provided on the tips and/or projections of the at least one body in particular the electrode fibers and/or electrode rods.

10. The cleaning device according to claim 1, wherein the second electrode extends along the first electrode at a defined distance, wherein a flow channel for the gas is formed by the defined distance between the first electrode and the second electrode.

11. The cleaning device according to claim 1, wherein at least one guide device preferably having a fan is provided, in order to guide the gas to be cleaned through the flow channel between the first electrode and the second electrode.

12. The cleaning device according to claim 1, wherein the plurality of tips and/or projections stand out from the first electrode in the direction of the second electrode with a length of between 0.5 and 40 mm, preferably 1 and 20 mm, particularly preferably 1 and 5 mm.

13. The cleaning device according to claim 1, wherein the plurality of tips and/or projections extend from the first electrode in the direction of the second electrode with different lengths.

14. The cleaning device according to at least claim 1, wherein the second electrode is formed by at least one liquid bath, preferably a water bath.

15. The cleaning device according to claim 1, wherein the cleaning device has a filtration device, preferably upstream of the electrodes in terms of flow.

16. The cleaning device according to claim 1, wherein the cleaning device has a heating device, preferably wherein the control or regulating device is formed to control or regulate a heating capacity of the heating device.

17. The cleaning device according to claim 16, wherein the heating device has at least one heating layer, which at least one heating layer is arranged on the tips and/or projections of the at least one body—in particular the electrode fibers and/or electrode rods and is formed to control the temperature of the tips and/or projections of the at least one body with the aid of the voltage applied to the first electrode.

18. The cleaning device according to claim 16, wherein the control or regulating device is formed to vary a heating capacity of the heating device, in particular of the at least one heating layer, taking into account a temperature and/or humidity and/or ozone content and/or level of loading and/or pressure of the gas—preferably using a temperature and/or ozone and/or particulate and/or pressure and/or humidity sensor connected or connectable to the control or regulating unit.

19. The cleaning device according to claim 1, wherein the cleaning device has at least one external dimension which corresponds to internationally standardized high-efficiency particulate air filters.

20. The cleaning device according to claim 1, wherein the cleaning device has a temperature control device downstream of the electrodes in terms of flow, preferably wherein the control or regulating device is formed to actuate or regulate the temperature control device in such a way that a gas temperature of the gas leaving the cleaning device can be set.

21. An arrangement of the cleaning device according to claim 1 in a ceiling panel and/or a wall covering and/or a floor panel and/or a couch and/or a chair, wherein the cleaning device is preferably integrated into a headrest and/or the gas to be cleaned can be fed to the cleaning device via a projection.

22. A use of the cleaning device according to claim 1 for cleaning a gas, preferably an ambient air, of at least one building interior, one vehicle interior, in enclosed spaces of vehicles, flying devices, watercraft, spacecraft cabins, space stations, lifts, medical practices, operating rooms, building interiors and/or interiors of medical facilities.

23. The use according to claim 22, wherein the cleaning device is used in a ceiling panel and/or a wall covering and/or in a freestanding and/or portable manner.
